(19) United States
(12) Patent Application Publication
(10) Pub. No.: US 2009/0005714 A1
(43) Pub. Date: Jan. 1, 2009

(54) Massage Device

(76) Inventor: Guerrino Giuseppe Mecenero, Huddersfield (GB)

Correspondence Address: ADAMS INTELLECTUAL PROPERTY LAW, P.A., Suite 2350 Charlotte Plaza, 201 South College Street, CHARLOTTE, NC, 28244, US

(21) Appl. No.: 12/097,289

(22) PCT Filed: Dec. 14, 2006
(86) PCT No.: PCT/GB06/04695

§ 371 Date: Sep. 9, 2008

(30) Foreign Application Priority Data
Dec. 15, 2005 (GB) 0525520.3
Dec. 14, 2006 (GB) PCT/GB2006/004695

(51) Int. Cl. A61H 1/00; A61H 7/00; A61F 5/00

(52) U.S. Cl. 601/46; 601/134; 600/38

(57) ABSTRACT

A massage device comprising: an inner sleeve; an outer sleeve; a composition sealed between the inner sleeve and the outer sleeve, the composition being capable of undergoing a phase change which is accompanied by a change in temperature of the composition. The exterior surface of the massage device is therefore able to be warmed to a desired operating temperature.

### FIELD OF THE INVENTION

The present invention relates to a massage device and a method of using such a device.

### BACKGROUND OF THE INVENTION

Vibrating massagers for sexual stimulation are well known. A problem associated with such massagers is that when they are cold, they feel uncomfortable when in contact with the human body

It is an aim of the present invention to overcome at least one problem associated with the prior art whether referred to herein or otherwise.

### SUMMARY OF THE INVENTION

According to the present invention there is provided an apparatus and method as set forth in the appended claims. Preferred features of the invention will be apparent from the dependent claims, and the description which follows.

According to a first aspect of the present invention there is provided a massage device comprising: an inner sleeve; an outer sleeve; a composition sealed between the inner sleeve and the outer sleeve, the composition being capable of undergoing a phase change which is accompanied by a change in temperature of the composition; and a means for inducing said phase change.

Preferably the massage device is a sexual massage device.

Preferably the inner sleeve is substantially tubular over the majority of its length and may be closed at one end. The inner sleeve is preferably made of silicone.

Preferably the outer sleeve is substantially tubular over the majority of its length and may be closed at one end.

Preferably the external surface the outer sleeve is smooth to slide over human skin.

Preferably the material of the outer sleeve has a heat conductivity such that the external surface of the outer sleeve is at a suitable temperature for contact with the human body.

Preferably, the outer sleeve is made of silicone.

Preferably the inner sleeve and the outer sleeve are bonded or welded together by means of a silicone based adhesive.

Preferably the inner sleeve is partially filled with a core material. The core material is preferably foam and may be silicone foam.

Preferably, the massage device further comprises a vibration generating means for causing the outer surface of the massage device to vibrate. The vibration generating means may be located at a first open end of the inner sleeve.

Preferably the vibration generating means is a motor. The motor may be removable. The motor may be fixed within the first open end of the inner sleeve by friction fit. The motor may be mains powered or battery operated.

A head may be located at a second end of the foam core. The head may be a filled gland and may be fitted by friction fit within the second end of the inner sleeve and extend out of the inner sleeve.

Any composition which undergoes a phase change which is accompanied by a change in temperature of the composition may be used. Preferably the phase change is accompanied by an increase in temperature of the composition. Preferably the increase in temperature is at least 5° C., and more preferably at least 10° C. Preferably the increase in temperature is up to 50° C., and more preferably up to 40° C.

Preferably the composition is a liquid and the phase change involves the change from a liquid to a solid. Most preferably the composition comprises an aqueous solution and the phase change is a crystallization, most preferably an exothermic crystallization.

Preferably the composition comprises a salt solution, preferably a salt solution which is super-saturated at room temperature. A super-saturated solution is a solution which comprises a solute in a concentration which is higher than the solubility limit of the solute at that temperature.

Preferably the composition comprises a supersaturated solution of a salt of an organic acid. Preferably the composition comprises an alkali metal salt of a carboxylic acid having 1 to 4 carbon atoms.

Most preferably the composition comprises a supersaturated solution of sodium acetate.

Methods for preparing such solutions are well known to those skilled in the art. The solution may be prepared, for example from food grade sodium acetate trihydrate.

Preferably the means for inducing a phase change comprises a thin ferrous metal strip in the form of a band around the tubular portion of the inner sleeve. The metal strip may have slits which rub together when the metal strip is pressed.

The means for inducing a phase change may comprise a concave metal disc. The metal disc may have slits which rub together when the metal disc is pressed. The metal disc may be located on the end of an elongate attachment means.

The means for inducing a phase change may be located in a head portion of the massage device. The means for inducing a phase change may be located within the solution by means of a holder, preferably comprising an elongate stem portion and a grommet portion for holding the means for inducing a phase change. The holder may support the means for inducing a phase change from the inner sleeve and is preferably made of silicone.

The means for inducing a phase change may alternatively be located externally of the inner and outer sleeves.

An opening may be provided in the inner or outer sleeve to enable the insertion of the composition. A plug may be provided for sealing the opening. The plug is preferably a friction fit within the opening. After insertion of the plug, adhesive may be applied to the opening to enhance the seal.

The inner sleeve or outer sleeve may be provided with an opening to enable insertion of the composition and a sealing means may be provided for sealing the opening. The sealing means may be a plug.

Preferably, the interior of the plug is hollow and is in fluid communication with the composition located between the inner and outer sleeves, and the means for inducing a phase change is located in the interior of the plug.

Preferably the massage device is recharged by heating the massage device until the crystallised solution returns to a liquid state. Heating the massage device may comprise placing the massage device in boiling water.

Preferably the inner or outer sleeve of the massage device may be provided with ridges to keep the outer membrane at a desired spacing from the inner sleeve.

Preferably the inner sleeve is provided with a reflective foil to reflect heat outwardly from the massage device. The foil may be gold foil. The foil may be located on the inner or outer surface of the inner sleeve.

The outer and inner sleeves may be flexible to allow the massage device to bend. The outer and inner sleeves may be provided with corrugations to enable the massage device to bend.

The massage device may comprise a further substantially tubular portion. The inner and outer sleeves may extend over the further substantially tubular portion.

According to a further aspect of the invention there is provided a dual ended massage device comprising a first massage device and a second massage device as herein referred to.

Preferably the first and second massage devices are separably connected, preferably by twist fit.

According to a further aspect of the invention there is provided an anal massage device according to any of the preceding claims.

According to a further aspect of the invention there is provided a method of operating a massage device when the massage device is as herein referred to.

According to a further aspect of the invention there is provided a method of manufacturing a massage device when the massage device is as herein referred to.

The present invention includes any combination of the herein referred to features or limitations.

### BRIEF DESCRIPTION OF THE DRAWINGS

For a better understanding of the invention, and to show how embodiments of the same may be carried into effect, reference will now be made, by way of example, to the accompanying diagrammatic drawings in which:

FIG. 1 is a cross sectional view through the length of a massage device according to a second embodiment of the present invention;

FIG. 2 is a cross section through the line 2-2 of the massage device of FIG. 1

FIG. 3 is a cross sectional view through the length of a massage device according to a second embodiment of the invention;

FIG. 4 is a cross sectional view through the length of a massage device according to a third embodiment of the present invention;

FIG. 5 is a cross sectional view through the length of an anal massage device according to a fourth embodiment of the present invention;

FIG. 6 is a cross sectional view through the length of a dual ended massage device according to a fifth embodiment of the invention;

FIG. 7 is an external view of a flexible massage device according to a sixth embodiment of the invention;

FIG. 8 is a cross-sectional view through the length of a massage device according to a seventh embodiment of the invention;

FIG. 9 is a partial perspective view through a section of the massage device of FIG. 8;

FIG. 10 is a cross-sectional view through the length of a massage device according to an eighth embodiment of the invention; and

FIGS. 11A and 11B are detailed views from above and the side of the plug of FIG. 10.

### DESCRIPTION OF THE PREFERRED EMBODIMENTS

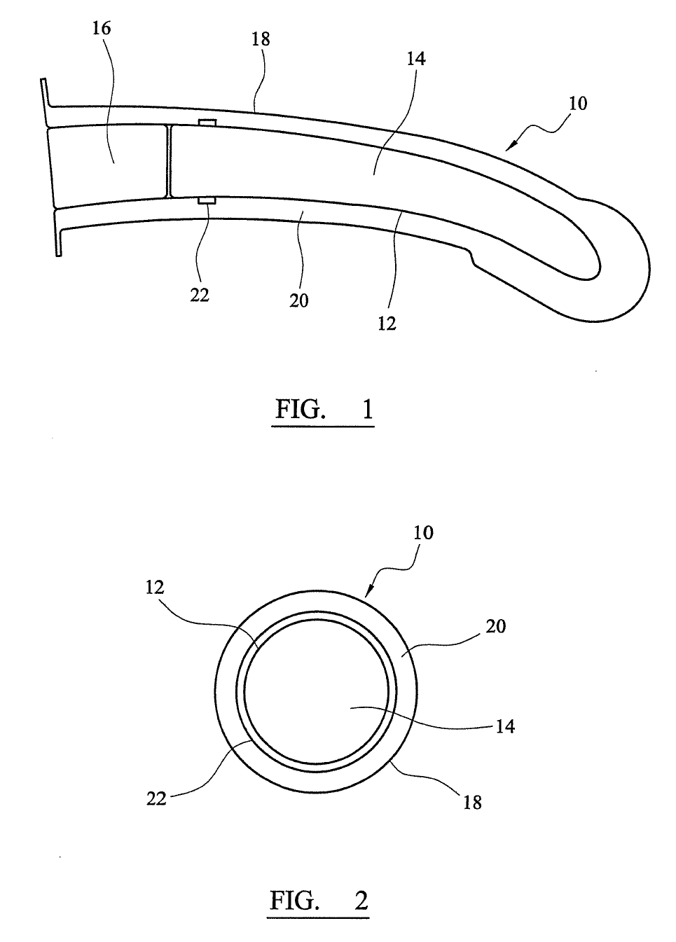

FIG. 1 shows a massage device 10 having a tubular inner sleeve 12 which is closed at one end. The inner sleeve 12 is preferably made of silicone. The inner sleeve 12 is partially filled at the closed end with foam to provide a rigid tubular core 14. A vibratory motor 16 is located by friction fit in an open end of the inner sleeve and abuts an end of the foam core 14. Silicone foam is preferably used for the core 14 as it is good transmitter of vibration. The vibratory motor 16 is a conventional motor capable of generating vibrations and which is either mains powered or battery operated.

The massage device 10 further comprises a tubular outer sleeve 18 which is closed at one end. The outer sleeve 18 is preferably made of silicone and is shaped to resemble a penis. The outer sleeve 18 and the inner sleeve 12 are bonded by means of a silicone based adhesive to form a sealed pocket for receiving a solution 20. The solution 20 is a saturated solution of sodium acetate that generates heat on crystallization. It should be noted that the sodium acetate is food grade and not toxic.

A triggering element 22 is provided comprising a thin ferrous metal band arranged in the solution 20 around the inner sleeve 12, as illustrated in FIG. 2. The metal band is flexible and is adapted to deform when pressed. The metal band is provided with slits (not shown), the sides of which rub against each other when the metal band is pressed.

In use, the liquid sodium acetate solution is initiated to start crystallization to generate heat by pressing the triggering element 22 so that the slits rub against each other. The triggering element 22 initiates crystallization by releasing minute crystals of solid sodium acetate trihydrate into the supersaturated solution 20. These crystals are harboured in submicron cracks on the surface of the triggering element 22 and are released when the triggering element 22 is flexed. The heat of crystallization causes an external temperature rise of 10° C. to 40° C. However, the temperature of the external surface of the outer sleeve 18 is also determined by the heat conductivity and thickness of the outer sleeve 18.

The sodium acetate solution 20 may be returned to its liquid state by immersion of the massage device 10 in boiling water. The immersion time is typically 15 minutes or more. However, the time required for immersion is determined by the need for every crystal to be melted, otherwise the liquid will re-solidify. Before placing the massage device 10 in boiling water, the motor 16 may be removed from the inner sleeve 12.

The phase change cycle of the solution 20 from solid to liquid and back again can be repeated as often as required.

Silicone is the preferred material for the inner sleeve 12 and the outer sleeve 18 because of its wide-range thermal stability, high lubricity, extreme water repellence, and physiological inertness. In use, the outer sleeve 18 will be in contact with the surface of the vagina, and therefore should be smooth and comfortable to the touch. Furthermore, the outer sleeve 18 should be capable of conducting heat from the solution 20 to the exterior surface of the outer sleeve 18, to maintain the exterior surface at a desired temperature.

Silicone foam is preferably used for the core 14 as it is good transmitter of vibration. However, any other suitable material may be used.

The inner sleeve 12 or outer sleeve 18 may be provided with ridges 4 mm in depth, 1.2 cm in width and 1.7 cm in length. The ridges would ensure that the outer sleeve 18 is kept at a desired spacing from the inner sleeve 12 and thus that the solution and the heat is evenly distributed around the massage device 10.

The manufacture of the massage device will now be described in more detail. The inner and outer sleeves 12, 18 are preformed to their respective shapes. The outer sleeve 18 is placed over the inner sleeve 12, and the inner sleeve 12 and the outer sleeve 18 are bonded by means of a silicone based adhesive around most of the circumference of their free ends, leaving a small opening. The inner sleeve 12 is then partially filled with foam to form the core 14. Next, the pocket between the inner sleeve 12 and outer sleeve 18 is filled with sodium acetate solution 20 via the opening. The opening is then bonded by means of a silicone based adhesive ensuring that no air bubbles remain in the solution 20. Air bubbles are to be avoided as they are detrimental to the performance of the sodium acetate solution.

The massage device 10 of the present invention could be provided with a reflective foil, such as gold foil, on the inner sleeve to reflect heat outwardly of the device. This would prevent the motor from overheating.

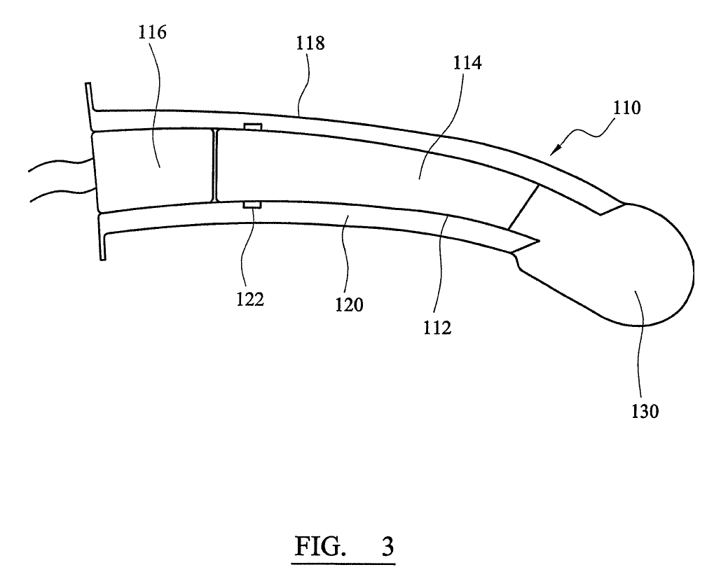

FIG. 3 shows a massage device 110 according to a second embodiment of the invention. The second embodiment is similar to the first embodiment and only the differences will therefore be described in detail.

The massage device 110 has a tubular inner sleeve 112, open at both ends. The inner sleeve 112 is partially filled with foam along a central portion to provide a rigid tubular core 114. A battery powered vibratory motor 116 is located by friction fit in a first end of the inner sleeve and abuts an end of the foam core 114.

The massage device 110 further comprises a tubular outer sleeve 118 open at both ends. The outer sleeve 118 is preferably made of silicone. The outer sleeve 118 and the inner sleeve 112 are sealed together with silicone based adhesive to form a sealed pocket for receiving a solution 120 as described previously. A triggering element 122 is provided, also as described previously.

The massage device further comprises a head 130 located at a second end of the foam core 114. The head 130 is a filled gland shaped to resemble a penis head. The head 130 is located by friction fit within a second end of the inner sleeve 112 and extends out of the inner sleeve 112.

FIG. 4 shows a massage device 210 according to a third embodiment of the invention. The third embodiment is similar to the first embodiment and only the differences will therefore be described in detail.

The massage device 210 comprises a first substantially tubular portion 230 and a smaller second substantially tubular portion 232, both of which are closed at one end. The first portion 230 is of a shape and dimensions to resemble a penis. The second portion 232 is designed to stimulate the external female genitals when the first portion 232 is inserted in the vagina.

The massage device 210 is provided with an outer sleeve 218 which is preferably made of silicone. The massage device 210 is also provided with an inner sleeve 212 which is also preferably made of silicone. A battery powered vibratory motor 216 is located by friction fit within in the inner sleeve 212. The motor 216 may alternatively be mains powered.

The outer sleeve 218 and the inner sleeve 212 are sealed together with silicone based adhesive to form a sealed pocket for receiving a solution 220 as described previously.

A triggering arrangement 224 is provided having a clicker 226 located in the head portion of the massage device 210. The clicker 226 is located within the solution 220 by means of a holder 227. The holder 227 comprises an elongate stem portion 228 and a grommet portion 229 for holding the clicker 226. The holder 227 supports the clicker 226 from the inner sleeve 212 and is preferably made of silicone.

The clicker 226 is a round concave metal sheet having a plurality of slits 227. The outer sleeve 218 is flexible enough that when the solution 220 is in a liquid state the clicker 228 may be operated. When the clicker 226 is pressed, it becomes concave in the opposite direction and in doing so the edges of the slits rub together to releasing minute crystals of solid sodium acetate trihydrate into the supersaturated solution 220.

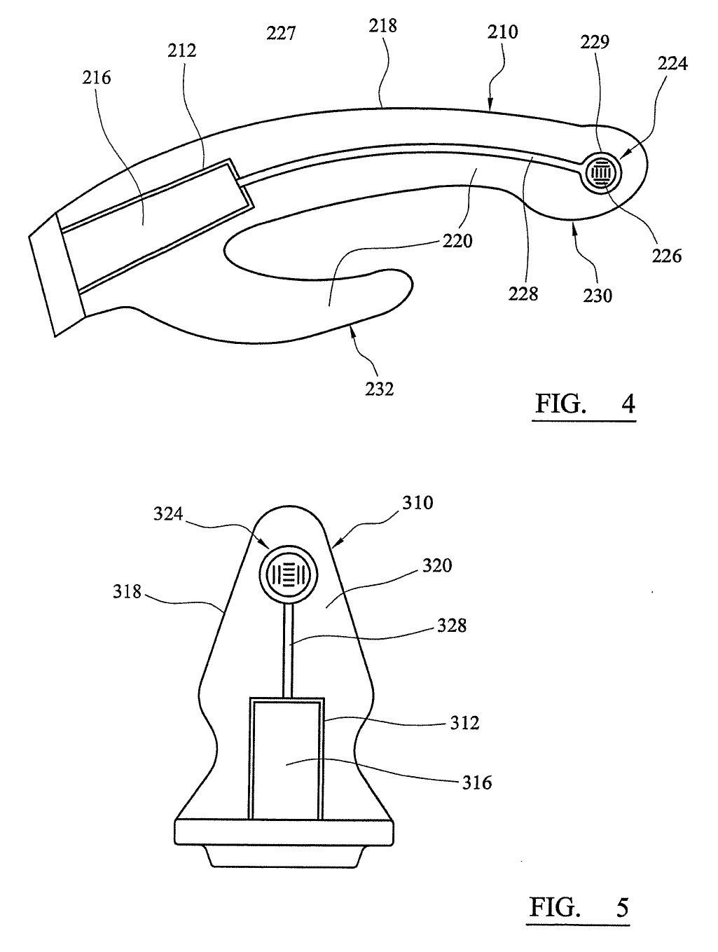

FIG. 5 shows a massage device 310 according to a fourth embodiment of the invention. The fourth embodiment is in the form of a butt plug for insertion into the anus. It is generally similar in construction to the third embodiment and only the differences will therefore be described in detail.

The massage device 310 is provided with a tubular outer sleeve 318 which is preferably made of silicone. The massage device 310 is also provided with a tubular inner sleeve 212 which is also preferably made of silicone. A battery powered vibratory motor 316 is located by friction fit within in the inner sleeve 312.

The outer sleeve 318 and the inner sleeve 312 are sealed together with silicone based adhesive to form a sealed pocket for receiving a solution 320 as described previously. A triggering arrangement 324 is provided, also as described previously.

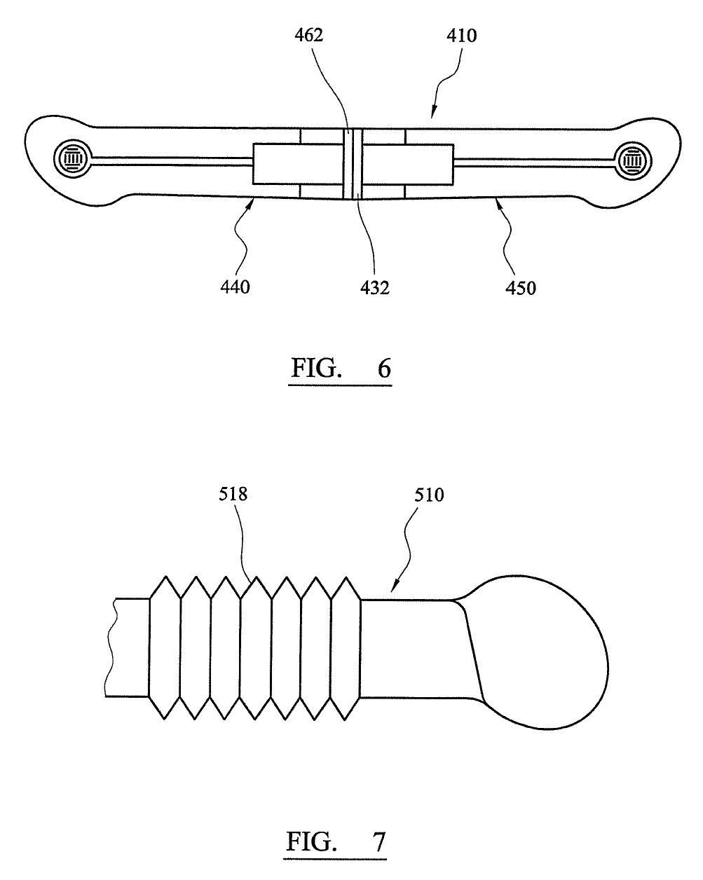

FIG. 6 shows a dual ended massage device 410 according to a fifth embodiment of the invention. This arrangement enables two women to use the device at the same time.

The dual ended massage device 410 comprises a first massage device 440 and a second massage device 450. Each massage device 440, 450 is generally similar in construction to previous embodiments and only the differences will therefore be described in detail.

The first and second massage devices 440, 450 are provided with an interlocking arrangement. This interlocking arrangement could, for example, be a twist fit arrangement or a friction fit arrangement. The interlocking arrangement 452,462 should be releasable to enable the motors to be removed so that the respective first and second massage devices can be safely placed in boiling water.

Furthermore, any of the massage devices described above may be constructed such that the outer and inner sleeves are sufficiently flexible to allow the massage device to bend. This flexibility may be obtained by providing part or all of the outer and inner sleeves with corrugations, as illustrated in the embodiment of FIG. 7.

FIG. 7 shows a massage device 510 having a corrugated portion of the outer sleeve 518. In the embodiments of FIGS. 1 to 3, the inner sleeve may also be corrugated.

The use of a motor in each of the embodiments is optional.

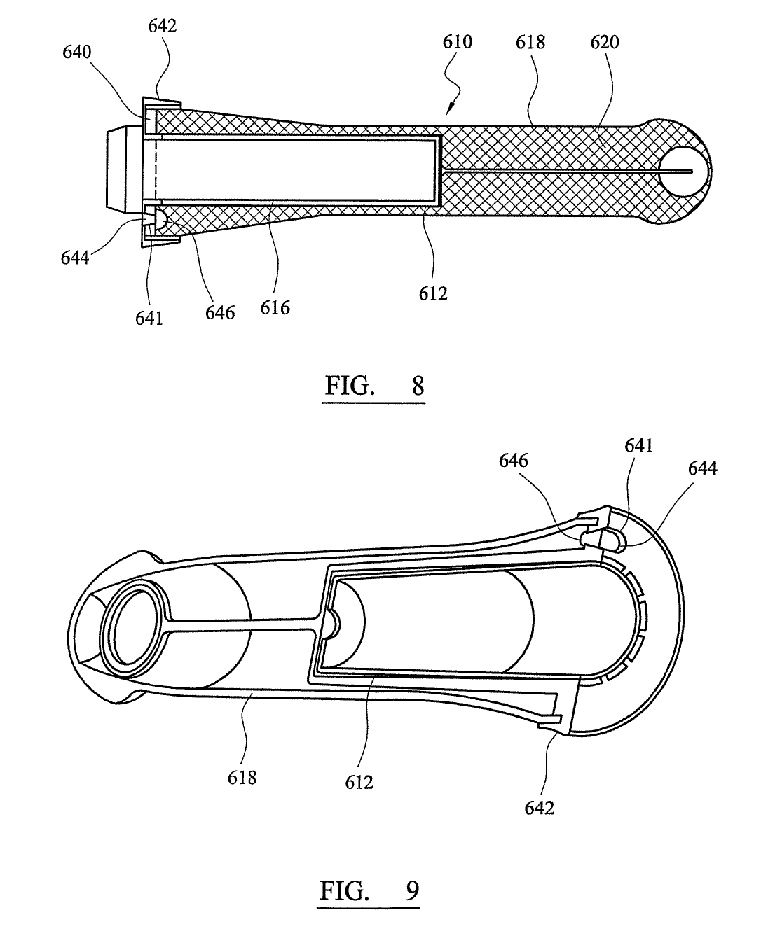

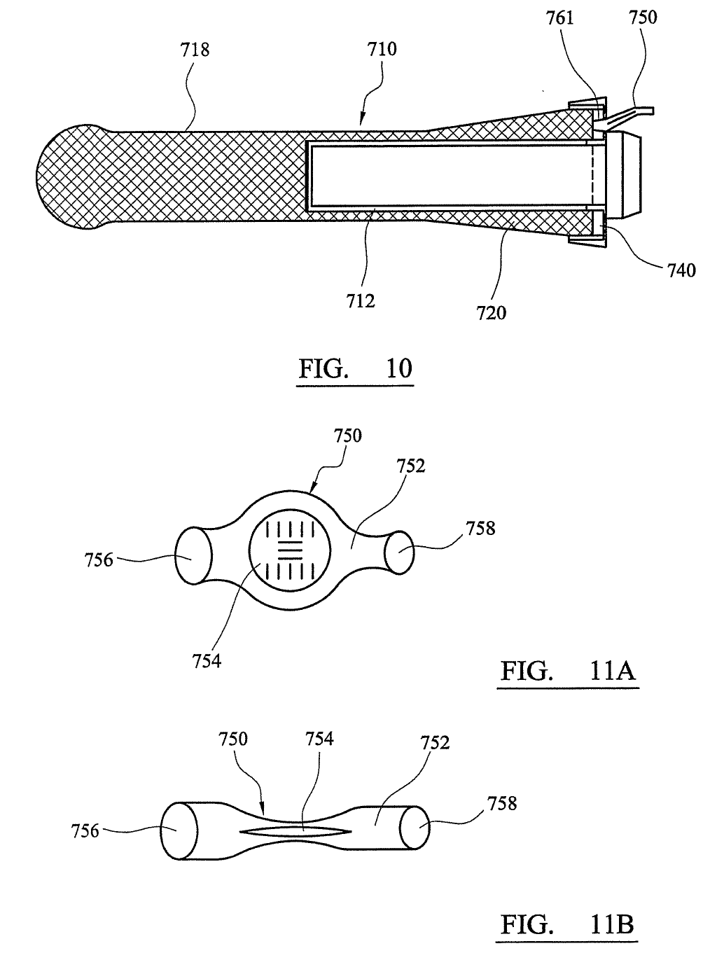

FIGS. 8 and 9 show a massage device 610 according to a seventh embodiment of the invention. It is generally similar in construction to the previous embodiments and only the differences will therefore be described in detail.

FIGS. 8 and 9 show in detail an arrangement for filling the massage device with a solution 620. The inner sleeve 612 is provided with a thickened base portion 640 of typically 9 mm. This base portion 640 is provided with an opening 641 through which the solution 620 is passed during manufacture. The opening 641 receives a plug 644 for sealing the solution 620 within the inner and outer sleeves 612, 618.

The plug 644 engages the wall of the opening 641 by friction fit to provide a seal. The plug 644 may also be provided with a head portion 646 which has a diameter larger than that of the opening 641 in order to enhance the seal. The elasticity of the plug 644 and/or the inner sleeve 612 enable the head portion 646 of the plug 644 to pass through the opening 641.

The plug 644 may be dimensioned to leave a small recess formed by the opening 641 on the outer surface of the base portion 640 when the plug 644 is fully inserted. This recess may be filled with adhesive to further ensure that a good seal is provided.

Alternatively the opening 641 may be provided in the outer sleeve.

The inner sleeve 612 may be reinforced as shown at 642. This reinforcement may either be integral with the inner sleeve 612 or may be a separate element. The reinforcement extends from inside the inner sleeve 612 in the region that receives the motor, along the base portion 640 of the inner sleeve 612 and around the outside of the outer sleeve 618.

FIG. 10 shows a massage device 710 according to a eighth embodiment of the invention. It is generally similar in construction to the embodiment of FIG. 8 and only the differences will therefore be described in detail.

FIG. 10 shows an arrangement for filling the massage device with a solution 720 and providing a triggering means. Plug 750 is made of silicone and is hollow, with a opening at each end 756, 758. The first end 756 may be larger than the second end 758. The first end 756 is adapted to be inserted in an opening 741 in a base portion 740 of the inner sleeve 712 as described previously. The interior of the plug is thus in fluid communication with the pocket formed by the inner and outer sleeves 712, 718. The second end 758 is designed so that it can be sealed to prevent solution 720 escaping from the massage device 710. Sealing may be achieved by folding over the second end 758 and sealing with silicone adhesive. Silicone adhesive may also be placed around the join between the inner sleeve 712 and the plug 750 to ensure a good seal.

The plug is provided with a clicker 754 to act as a triggering element as described previously. The clicker is completely immersed in the solution 720.

During manufacture the plug 750 is inserted in the inner sleeve 712 and the massage device filled with solution until the plug 750 is almost full. The second end 758 of the plug is then sealed, such that substantially no air remains in the plug 750.

Although a few preferred embodiments have been shown and described, it will be appreciated by those skilled in the art that various changes and modifications might be made without departing from the scope of the invention, as defined in the appended claims.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A massage device comprising: an inner sleeve; an outer sleeve; a composition sealed between the inner sleeve and the outer sleeve, the composition being capable of undergoing a phase change which is accompanied by a change in temperature of the composition; and a means for inducing the phase change.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A massage device according to claim 1 wherein the massage device further comprises a vibration generating means for causing the outer surface of the massage device to vibrate, the vibration generating means being located at a first open end of the inner sleeve.

16. (canceled)

17. A massage device according to claim 15, wherein the vibration generating means is a motor which is removable.

18. (canceled)

19. A massage device according to claim 17, wherein the motor is fixed within the first open end of the inner sleeve by friction fit.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A massage device according to claim 1 wherein the means for inducing a phase change comprises a concave metal disc.

44. A massage device according to claim 43, wherein the metal disc has slits which rub together when the metal disc is pressed.

45. A massage device according to claim 43, wherein the metal disc is located within the composition on the end of an elongate attachment means.

46. A massage device according to claim 45, wherein the elongate attachment means comprises an elongate stem portion and a grommet portion for holding the means for inducing a phase change.

47. (canceled)

48. (canceled)

49. (canceled)

50. (canceled)

51. (canceled)

52. (canceled)

53. (canceled)

54. (canceled)

55. A massage device according to claim 45, wherein the elongate attachment means supports the means for inducing a phase change from the inner sleeve.

56. (canceled)

57. (canceled)

58. (canceled)

59. (canceled)

60. A dual ended massage device comprising a first massage device and a second massage device according to claim 1.

61. (canceled)

62. (canceled)

63. (canceled)

64. A method of operating a massage device comprising the steps of:
(a) providing a massage device comprising: an inner sleeve, an outer sleeve, a composition sealed between the inner sleeve and the outer sleeve, the composition being capable of undergoing a phase change which is accompanied by a change in temperature of the composition, and a means for inducing the phase change;
(b) positioning the massage device near a person; and
(c) activating the means for inducing the phase change.

65. A method of manufacturing a massage device, comprising the steps of:
(a) providing an inner sleeve, an outer sleeve, and a composition capable of undergoing a phase change which is accompanied by a change in temperature of the composition;
(b) positioning the composition within the outer sleeve;
(c) positioning the inner sleeve within the composition; and
(d) sealing the outer sleeve to form a sealed massage device.

66. A massage device according to claim 45, wherein the elongate attachment means is preferably made of silicone.

67. A massage device according to claim 1, wherein the means for inducing a phase change is located in a head portion of the massage device.
